(19) United States
(12) Patent Application Publication
(10) Pub. No.: US 2023/0143793 A1
(43) Pub. Date: May 11, 2023

(54) COSMETIC COMPOSITIONS

(71) Applicant: TwinMedix LLC, Tampa, FL (US)

(72) Inventors: Michelle Shaffer, Tampa, FL (US); Tammy Fleming, Tampa, FL (US)

(21) Appl. No.: 17/916,750

(22) PCT Filed: Apr. 1, 2021
(86) PCT No.: PCT/US2021/025383

§ 371 Date: Oct. 3, 2022

Related U.S. Application Data
(60) Provisional application No. 63/004,025, filed on Apr. 2, 2020.

(51) Int. Cl. A61K 8/67; A61Q 19/08; A61K 8/81

(52) U.S. Cl. CPC A61K 8/671; A61Q 19/08; A61K 8/8152; A61K 2800/95; A61K 2800/30

(57) ABSTRACT

Disclosed herein are cosmetic compositions that provide for an improved appearance of the dermis and for improved dermal and epidermal health in view of conventional formulations.

### TECHNICAL FIELD

The present invention relates generally to the field of cosmetics, and more particularly, to cosmetic compositions for topical use for improved appearance of the dermis and for improved dermal and epidermal health.

### SUMMARY

It is an object of the invention to provide various cosmetic compositions as disclosed herein that provide for an improved appearance of the dermis and for improved dermal and epidermal health in view of conventional formulations.

Additional features, aspects and advantages of the invention will be set forth in the detailed description which follows, and in part will be readily apparent to those skilled in the art from that description or recognized by practicing the invention as described herein. It is to be understood that both the foregoing general description and the following detailed description present various embodiments of the invention, and are intended to provide an overview or framework for understanding the nature and character of the invention as it is claimed.

### DETAILED DESCRIPTION

The present invention will now be described more fully hereinafter. In this specification and in the claims that follow, reference will be made to a number of terms that shall be defined to have the following meanings: It must be noted that, as used in the specification and the appended claims, the singular forms “a,” “an” and “the” include plural referents unless the context clearly dictates otherwise. “Optional” or “optionally” means that the subsequently described event or circumstance can or cannot occur, and that the description includes instances where the event or circumstance occurs and instances where it does not. As used herein, the term “about” is used to provide flexibility to a numerical range endpoint by providing that a given value may be “slightly above” or “slightly below” the endpoint without affecting the desired result. Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within the ranges as if each numerical value and sub-range is explicitly recited. As an illustration, a numerical range of “about 1 to 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc. as well as 1, 2, 3, 4, and 5, individually. The same principle applies to ranges reciting only one numerical value as a minimum or a maximum. Furthermore, such an interpretation should apply regardless of the breadth of the range or the characteristics being described. The compositions and methods described herein can comprise, consist of, or consist essentially of the essential elements and limitations described herein, as well as any additional or optional ingredients, components, or limitations described herein. It is understood that any given particular aspect of the disclosed compositions and methods can be easily compared to the specific examples and embodiments disclosed herein. By performing such a comparison, the relative efficacy of each particular embodiment can be easily determined. Particularly preferred compositions and methods are disclosed in the Examples herein, and it is understood that these compositions and methods, while not necessarily limiting, can be performed with any of the compositions and methods disclosed herein.

### Formulation 1: Ascorbic Acid/Vitamin C Based Topical Composition

In certain aspects disclosed is an ascorbic acid/vitamin C based topical formulation that inhibits and reduces endogenous enzymatic activity produce melanin, which leads to hyperpigmentation and/or a hyperpigmented phenotype related to inflammatory process associated with skin damage (for excessive sun exposure) and age. In certain aspects, this formulation inhibits and/or reduces endogenous matrix metalloproteinase (MMPs) that are associated with breaking down collagen and further leading to hyperpigmentation, thereby reducing hyperpigmentation while concurrently and advantageously providing a firmerlifted appearance to the skin by preventing and/or limiting the breakdown of endogenous collagen.

### Quali-Quantitative Formulation Chart

| | | INGREDIENT | |
|---|---|---|---|
| % (w/w) | | (INCI NAME) | |
| Range | CAS No. | LISTED IN ICID | FUNCTION/s |
| 70.0-90.00 | 541-02-6 | Cyclopentasiloxane | Emollient, Solvent |
| 10.0-20.0 | n/a | Dimethicone | Viscosity |
| | | Crosspolymer | controlling |
| 10.0 | 50-81-7 | Ascorbic | Anti-oxidant, |
| | | Acid | skin conditioning |
| 1.0-5.0 | 25777-71-3 | Methyl | Film forming |
| | | Methacrylate/Glycol | |
| | | Dimethacryalte | |
| | | Crosspolymer | |
| 0.10-0.99 | 1406-18-4 | Tocopherol | Anti-oxidant |
| 0.10-0.99 | 90106-37.9 | Oryza Sativa | Skin conditioning |
| | | (Rice) Bran Extract | |
| 0.10-0.99 | 84776-03-4 | Helianthus Annuus | Skin conditioning |
| | | (Sunflower Extract) | |
| 0.10-0.99 | 84604-14-8 | Rosmarinus Officinalis | Skin conditioning |
| | | (Rosemary) | |
| | | Leaf Extract | |

In certain aspects, the ascorbic acid/vitamin C based topical formulation includes a volatile inorganic solvent that also functions as an emollient that evaporates upon application to a user and further facilitates film formation of the film forming matrix components (discussed further below). In certain aspects, the volatile inorganic solvent is ranges from 65 wt % to 95 wt % of the formulation, more preferably from 68 wt % to 92 wt % of the formulation, and most preferably from 70 wt % to 90 wt % of the formulation. In certain aspects, the volatile inorganic solvent is siloxane based, and more preferably, a cyclic siloxane. For example, cyclic siloxanes generally exhibit good solvation and volatility characteristics in siloxanes, and their participation in matrix formation is generally relatively low due to the fact that all silane oxygen atoms are unavailable polymerization. Thus, the siloxane is not included within the film forming matrix (discussed further below) within this formulation. As shown in the table above, the cyclic siloxane is preferably cyclopentasiloxane ranging from 65 wt % to 95 wt % of the formulation, more preferably from 68 wt % to 92 wt % of the formulation, and most preferably from 70 wt % to 90 wt % of the formulation. The volatile component (cyclopentasiloxane) is preferably present in amounts such that the volatile component is more than 50 percent evaporated after 15 minutes at one or more temperatures in the range of from about 30° C. (86° F.) to 40° C. (104° F.).

in certain aspects, the ascorbic acid/vitamin C based topical formulation further includes a film forming matrix comprised of at least one crosslinking agents and reactive acrylic acids (or the salts thereof) (pre-polymers that react and polymerize upon application on the user). These components react with one another to form a film upon application to a person's skin while the volatile inorganic solvent evaporates away from th.e skin and remains non-reactive with the film forming matrix. For example, in certain aspects, the at least one crosslinking agents and reactive acrylic acids (or the salts thereof) includes a methacrylic acid (or methacrylate) and an acrylic based (or acrylate based) crosspolymer, with the total amount of both ranging from 0.5 wt % to 7.5 wt % of the overall formulation or more preferablyl wt % to 5 wt % of the overall formulation. In certain aspects, the methacrylic acid (or methacrylate) and an acrylic based (or acrylate based) crosspolymer are methyl methacrylate and glycol dimethacrylate crosspolymer respectively, with the total amount of both ranging from 0.5 wt % to 7.5 wt % of the overall formulation or more preferably I wt % to 5 wt % of the overall formulation.

It is preferable to control and maintain operable viscosity of the formulation during storage at ambient temperature(s) (i.e., storage conditions ranging from 40° F. to 120° F.). In order to do so, viscosity controlling agents are included. In the formulation. For example, the inclusion of, for example, a dimethicone crosspolymer provides a composition with a thixotropic consistency. The dimethicone crosspolymer is preferably included within the formulation at a concentration ranging from 15 wt % to 25 wt % of the formulation, from 12.5 wt % to 22.5 wt % of th.e formulation, or most preferably from 10 wt % to 20 wt % of the formulation. The dimethicone crosspolymer further aids in crosslinking and film formation of the film forming matrix when the formulation is applied to an individual and the volatile inorganic solvent (cyclopentasiloxane) evaporates away.

Further included within the ascorbic acid/vitamin C based topical formulation is a primary antioxidant/primary active agent. The primary antioxidant is Vitamin C/Ascorbic Acid at a concentration range from 8 wt % to 12 wt %, and most preferably 9.5 wt % to 10.5wt %. These ranges are preferred so that adequate migration of the Vitamin C/Ascorbic acid occurs through the film formed (by the film forming matrix) on the skin occurs and is absorb by the individual's skin from the film. If concentrations of Vitamin C/Ascorbic Acid are decreased below 8 wt % inadequate migration of the Vitamin C/Ascorbic Acid through the film occurs thus resulting in inadequate skin absorption, which further results in an inadequate reduction of MMP activity, hyperpigmentation, and collagen breakdown. However, if concentrations of Vitamin C/Ascorbic Acid are increased above 12 wt %, no marked improvement of decreased MMP activity, hyperpigmentation, and collagen has been observed. In certain aspects, a secondary antioxidant/secondary active agent is further included in the formulation. This secondary antioxidant/secondary active agent synergistically acts with the primary antioxidant/primary active agent (Vitamin C/Ascorbic Acid) to reduce MMP activity, hyperpigmentation, and collagen breakdown thereby improving the appearance of the user's skin. In certain aspects, the secondary antioxidant/secondary active agent is present at a concentration ranging from 0.05 wt % to 1.5 wt % of the composition and more preferably from 0.10 wt % to 1 wt % of the composition. The secondary antioxidant/secondary active agent preferably includes tocopherol or a salt thereof, which includes at least one, at least two, at least three, or at least all of the following: alpha-tocopherol, beta-tocopherol, gamma-tocopherol, and delta-tocopherol. In certain aspects, the secondary antioxidant/secondary active agent preferably includes tocopherol or a salt thereof at a concentration ranging from 0.05 wt % to 1.5 wt % of the composition and more preferably from 0.10 wt % to 1 wt % of the composition

In addition to the above, skin conditioners can be included to further soften the user's skin and to make use of the formulation more desirable. For example, in certain aspects, at least one of, at least two of, or all of the following may include: Rice Bran Extract (Oryza Sativa Bran Extract), Sunflower Extract (Helianthus Anuus Extract), and/or Rosemary extract (Rosmarinus Officinalis Leaf Extract) with each of these extracts, when present, having concentrations ranging from 0.05 wt % to 1.5 wt % of the composition and more preferably from 0.10 wt % to 1 wt % of the composition.

In certain aspects, the ascorbic acid vitamin C based topical formulation is paraben-free, fragrance-free, and gluten-free.

### Formulation 2: Aloe-Based Exfoliating Composition

Also disclosed herein is an aloe-based exfoliating composition. In certain aspects, this exfoliating composition is a liquid at ambient temperatures and includes the water-based aloe barbadensis leaf juice as the primary chemical component at concentrations ranging from 65 wt % to 95 wt % of the composition and more preferably 70 wt % to 90 wt % of the composition. Unlike, other exfoliating compositions, which are alcohol based and/or have high concentrations of alcohols (e.g., in excess of 15 wt %), the aloe-based exfoliating composition is a water based composition with minimal amounts of alcohol(s) included therein (i.e., not exceeding 10 wt %, 7.5 wt %, 5 wt %, 4 wt %, 3 wt %, 2 wt %, or 1 wt %). This exfoliating composition. abrades and removes excessive epidermal cells from a user via an exfoliating agent included therein while concurrently reducing inflammation via the presence of the aloe barbadensis leaf juice along with the skin conditioners and antioxidant (discussed further below). In certain aspects, the exfoliating agent is sodium chloride, and more particularly, the exfoliating agent is sea salt that is either partially or completely dissolved in the exfoliating composition. In certain aspects, the exfoliating agent is present at concentrations ranging from 0.0001 wt % to 1.5 wt % and more preferably from 0.0001 wt % to 0.99 wt % in the exfoliating composition. In certain aspects, the exfoliating agent is preferably only partially dissolved within the composition so that the exfoliating agent may physically abrade the user's skin surface to further enhance excessive epidermal cell removal and the smoothness of the user's skin.

### Quali-Quantitative Formulation Chart

| | | INGREDIENT | |
|---|---|---|---|
| % (w/w/) | | (INCI NAME) | |
| Range | CAS No. | LISTED IN ICID | FUNCTION |
| 70.0-90.0 | 85507-69-3 | Aloe Barbadensis Leaf Juice | Skin conditioning |
| 1.0-5.0 | 9005-64-5 | Polysorbate 20 | Emulsifying, |
| | | | surfactant |
| 1.0-5.0 | 7732-15-5 | Water (Aqua) | Solvent |
| 1.0-5.0 | 56-81-5 | Glycerin | Humectant |
| 1.0-5.0 | 57-13-6 | Urea | Skin conditioning |
| 1.0-5.0 | 9007-20-9/9003-01-4/ | Carbomer | Gel forming, |
| | 76050-42-5/9062-04-8/ | | viscosity |
| | 9007-16-3/9007-17-4 | | controlling |
| 1.0-5.0 | 112-02-7 | Cetrimonium Chloride | Emulsifying, |
| | | | surfactant |
| 0.0001-0.99 | 8001-31-8 | Cocos Nucifera | Skin conditioning |
| | | (Coconut) Fruit Extract | |
| 0.0001-0.99 | 84650-00-0 | Coffea Arabica | Skin conditioning |
| | | (Coffee) Seed Extract | |
| 0.0001-0.99 | 90028-06-1 | Dipteryx Odorata Seed Extract | Skin conditioning |
| 0.0001-0.99 | 68650-44-2 | Prunus Armeniaca | Skin conditioning |
| | | (Apricot) Fruit Extract | |
| 0.0001-0.99 | 85251-63-4 | Pyrus Malus | Skin conditioning |
| | | (Apple) Fruit Extract | |
| 0.0001-0.99 | 8024-06-4/84650-63-5 | Vanilla Planifolia Fruit Extract | Skin conditioning |
| 0.0001-0.99 | 7647-14-5 | Sea Salt | Exfoliating |
| 0.0001-0.99 | 7695-91-2 | Tocopheryl Acetate | Anti-oxidant |
| 0.0001-0.99 | 84650-60-2 | Camellia Sinensis Leaf Extract | Skin conditioning |
| 0.0001-0.99 | 73398-61-5/65381-09-/ | Caprylic/Capric Triglyceride | Skin conditioning |
| 0.0001-0.99 | 68937-54-2 | PEG-12 Dimethicone | Skin conditioning |
| 0.0001-0.99 | 139-33-3/6381-92-6 | Disodium EDTA | Chelating |
| 0.1125 | 70445-33-9 | Ethlhexylglycerin | Skin conditioning, |
| | | | part of Preservative |
| 0.2250 | 1117-86-8 | Caprylyl Glycol | Skin conditioning, |
| | | | part of Preservative |
| 0.1125 | 107-41-5 | Hexylene Glycol | Solvent, part of |
| | | | Preservative |
| 0.4500 | 122-99-6 | Phenoxyethanol | Preservative |

To further preserve moisture content and reduce inflammation of the user's skin while using exfoliating composition, one or more humectants are included within the composition. In certain aspects, the humectant is a glyceride (lipid) derivative and is preferably glycerin (glycerol), which is present in the composition at a concentration ranging from 0.5 wt % to 7.5 wt % and more preferably from 1 wt % to 5 wt %. To solubilize the humectant (lipid based humectant) as well as any other lipid-based extracts/skin conditions (discussed further below) within the exfoliating composition, an emulsifying agent may be further included at a concentration of 0.5 wt % to 10 wt % of the composition and more preferably from 1 wt % to 5 wt % of the composition, which maintiains the chemical components in a mixed state (i.e., no visible phase separation) at ambient temperatures. In certain aspects, the emulsifying agent is a quaternary ammonium compound included at a concentration of 0.5 wt % to 10 wt % of the composition and more preferably from 1 wt % to 5 wt % of the composition and is more preferably cetrimonium chloride included at a concentration of 0.5 wt % to 10 wt % of the composition and more preferably from 1 wt % to 5 wt % of the composition.

As discussed above, the exfoliating composition is water based and preferably includes water as the solvent. In certain aspects, water is included at a concentration ranging from 0.5 wt % to 10 wt % of the composition and more preferably from 1 wt % to 5 wt % of the composition.

Further included in the exfoliating composition is preferably a water soluble antioxidant that is present in the composition at a concentration ranging from 0.0001 wt % to 2.0 wt % of the composition and more preferably from 0.0001 wt % to 1 wt % of the composition, which reduces inflammation of the user's skin while using exfoliating composition. The water soluble antioxidant is preferably tocopherol or a salt thereof (tocopherol acetate), which includes at least one, at least two, at least three, or at least all of the following: alpha-tocopherol (or the salt thereof), beta-tocopherol (or the salt thereof), gamma-tocopherol (or the salt thereof), and delta-tocopherol (or the salt thereof). In certain aspects, tocopherol or the salt thereof is included at a concentration ranging from 0.0001 wt % to 2.0 wt % of the composition and more preferably from 0.0001 wt % to 1 wt % of the composition.

In addition to the above, skin conditioners are further included in the exfoliating composition to further soften the user's skin and to make use of the composition more desirable while concurrently conditioning and reducing potentially inflammation of the user's skin that may be associated with the use of the exfoliating composition. For example, in certain aspects, the exfoliating composition includes urea as a first skin conditioner that is present at a concentration ranging from 0.5 wt % to 7.5 wt % of the composition and more preferably from 1 wt % to 5 wt % of the composition. In addition to urea additional skin conditioners may be included within the exfoliating composition. For example, at least one of, at least two of, at least three of, at least four of, at least five of, at least six of, at least seven of, at least eight of, or all of the following may include: coconut extract (cocas nucifora fruit extract), coffee extract (coffea Arabica seed extract), dipteryx odorata seed extract, apricot extract (prunus armeniaca fruit extract), apple extract (pyrus malas fruit extract), vanilla planifolia fruit extract, camellia sinensis leaf extract, caprylicicapric triglyceride, and PEG-12 dimethicone, with each of these extracts, when present, having concentrations ranging from 0.05 wt % to 1.5 wt % of the composition and more preferably from 0.10 wt % to 1 wt % of the composition.

In certain aspects, chelating agents and preservatives may be included to further improve the shelf life of the exfoliating composition. In particular, the chelating agent may include ethylenediaminetetraacetic acid (EDTA) at concentrations not exceeding 2 wt % and more preferably 1 wt % of the composition. For example, EDTA may be included in the composition at concentrations ranging from 0.0001 to 1 wt % and functions to bind any metallic contaminates that may be present in the composition, thus rendering such contaminates inert/innocuous. The preservatives included within the composition may include one or more polyols, which include, but are not limited to, a glycol or glycol derivative or a glycerol or glycerol derivative. In certain aspects, the preservatives include any combination of the following: ethylhexylglycerin, caprylyl glycol, hexylene glycol, and phenoxyethanol, wherein each of these preservatives, when present, range from 0.1 wt % to 0.8 wt % of the composition, and more preferably from 0.1 wt % to 0.5 wt % of the composition.

In certain aspects, the exfoliating composition is paraben-free, fragrance-free, and gluten-free.

### Foaming Composition for Topical Use

Also, disclosed herein is a foaming composition for topical use/topical application that cleanses the user's skin and pores by removing impurities therefrom. An exemplary embodiment of the foaming composition for topical rise/topical application is provided immediate below.

### Quali-Quantitative Formulation Chart

| | | INGREDIENT | |
|---|---|---|---|
| % (w/w) | | (INCI NAME) | |
| Range | CAS No. | LISTED IN ICID | FUNCTION/S |
| 50.0-70.0 | 7732-18-5 | Water (Aqua) | Solvent |
| 10.0-20.0 | 2235-54-3 | Ammonium Lauiryl Sulfate | Cleansing, surfactant |
| 10.0-20.0 | 355-42-0 | Perflurohexane | Solvent |
| 1.0-5.0 | 9005-64-5 | Polysorbate 20 | Emulsifying, surfactant |
| 1.0-5.0 | 107-88-0 | Butylene Glycol | Humectant |
| 1.0-5.0 | 27924-99-8 | Polyhydroxystearic Acid | Emulsifying |
| 1.0-5.0 | 59219-71-5 | Isononyl Isononanoate | Emollient, |
| | | | skin conditioning |
| 1.0-5.0 | 83682-78-4 | Sodium Cocamidopropyl | Foam boosting, surfactant |
| | | PG-Dimonium Chloride Phosphate | |
| 0.0001-0.99 | 24292-52-2 | Hesperidin Methyl Chalcone | Anti-oxidant |
| 0.0001-0.99 | n/a | Palmitoyl Tetrapeptide-7 | Skin conditioning |
| 0.0001-0.99 | n/a | Dipeptide-2 | Skin conditioning |
| 0.0001-0.99 | 84650-60-2 | Camellia Sinensis Leaf Extract | Skin conditioning |
| 0.0001-0.99 | 84961-57-9 | Punica Granatum Extract | Skin conditioning |
| 0.0001-0.99 | 84787-69-9 | Rubus Fruticosus | Skin conditioning |
| | | (Blackberry) Fruit Extract | |
| 0.0001-0.99 | n/a | Coffea Robusta Seed Extract | Skin conditioning |
| 0.0001-0.99 | 91770-88-6 | Vaccinium Macrocarpon | Skin conditioning |
| | | (Cranberry) Fruit Extract | |
| 0.0001-0.99 | 84929-27-1 | Vitis Vinifera (Grape) Leaf Extract | Skin conditioning |
| 0.0001-0.99 | n/a | Euterpe Oleraccea Fruit Extract | Skin conditioning |
| 0.0001-0.99 | 84776-10-3 | Arctostaphylos Uva Ursi | Skin conditioning |
| | | Leaf Extract | |
| 0.0001-0.99 | 85085-46-7 | Lycium Barbarum Fruit Extract | Skin conditioning |
| 0.0001-0.99 | 84929-68-0 | Morinda Citrifolia Fruit Extracdt | Skin conditioning |
| 0.0001-0.99 | 84929-76-0 | Rubus Idaeus (Raspberry) Fruit | Skin conditioning |
| 0.0001-0.99 | n/a | Brassica Oleracea Acephala | Skin conditioning |
| | | Leaf Extract | |
| 0.0001-0.99 | n/a | Aristotelia Chilensis Fruit Extracdt | Skin conditioning |
| 0.0001-0.99 | 56-81-5 | Glycerin | Humectant |
| 0.0001-0.99 | 9005-00-9 | Steareth-20 | Emulsifying |
| 0.0001-0.99 | 71566-49-9 | Ethylhexyl Isononanoate | Emollient |
| 0.0001-0.99 | 11138-66-2 | Xanthan Gum | Gel foaming, |
| | | | Emulsion stabilising |
| 0.1250 | 70445-33-9 | Ethylhexylglycerin | Skin conditioning, part of |
| | | | Preservative |
| 0.5000 | 122-99-6 | Phenoxyethanol | Preservative |
| 0.2500 | 1117-86-8 | Caprylyl Glycol | Skin conditioning, part of |
| | | | Preservative |
| 0.1250 | 107-41-5 | Hexylene Glycol | Solvent, part of |
| | | | Preservative |

As shown above, the foaming composition is water based in which water is the primary solvent and ranges from 65 wt % to 95 wt % of the composition and more preferably 70 wt % to 90 wt % of the composition. In certain aspects and to further aid with solubilizing various components included within the foaming composition, an organic solvent is included as the secondary solvent. In certain aspects, the organic solvent is preferably a fluorocarbon, and in certain aspects, the organic solvent is perfluorohexane at a concentration range from 8.5 wt % to 22.5 wt % and more preferably from 10 wt % to 20 wt % of foaming composition. Perfluorohexane is included within this composition because of its capability to dissolve gases, especially oxygen, at a higher concentration than ordinary organic solvents. This effect is attributed to the weak intermolecular forces between perfluorohexane molecules, which allows “space” for gas molecules to partition into the liquid. Perfluorohex.ane aids in delivering dissolved oxygen to the person's skin surface, which aids in further cleaning the person's skin surface and pores. In addition, the foaming composition further includes a detergent that functions to clean the surface of the user's skin. The detergent is included at a concentration range from 8.5 wt % to 22.5 wt % and more preferably from 10 wt % to 20 wt % of foaming composition. The detergent preferably is ammonium lauryl sulfate at a concentration range from 8.5 wt % to 22.5 wt % and more preferably from 10 wt % to 20 wt % of foaming composition.

To further preserve moisture content of the user's skin while using this composition, one or more humectants are included within the composition. In certain aspects, the humectant is a polyol that includes for example glycerin and butylene glycol. Butylene glycol is present in the composition at a concentration ranging from 0.5 wt % to 7.5 wt % and more preferably from 1 wt % to 5 wt % of the composition, and glycerin is present in the composition at a concentration ranging from 0.0001 wt % to 1.5 wt % and more preferably from 0.0001 wt % to 1. wt % of the composition. To solubilize the humectant(s) (lipid based humectant(s)) as well as any other lipid-based extracts/skin conditioners (discussed further below) within this composition, one or more emulsifying agent(s) having amphiphilic properties may be further included at a concentration of 0.5 wt % to 12.5 wt % of the composition and more preferably from 2 wt % to 11.5 wt % of the composition, which maintains the chemical components in a miscible, mixed state (i.e., no visible phase separation) at ambient temperatures. In certain aspects, the emulsifying agent includes a first, second, and third emulsifying agent wherein the first emulsifying agent is polysorbate 20 at a concentration of 0.5 wt % to 6.5 wt % of the composition and more preferably from 1 wt % to 5 wt % of the composition; the second emulsifying agent is polyhydroxystearic acid at a concentration of 0.5 wt % to 6.5 wt % of the composition and more preferably from 1 wt % to 5 wt % of the composition, and the third emulsifying agent is stearth-20 at a concentration of 0.0001 wt % to 1 wt % of the composition. Foaming agents may be further included within this composition that further aid with both foaming of the composition and further solubilizing the various lipophilic (lipid based) components. For example, the foaming agents may include sodium cocamidopropyl PG-Dimonium Chloride Phosphate at a concentration ranging from 0.5 wt % to 6.5 wt % of the composition and more preferably from 1 wt % to 5 wt % of the composition.

In addition various skin conditioning agents may be further included in the foaming composition. For example, any combination of skin conditioning agents selected from the following may be included in the foaming composition: isononyl isononanoate, palmitoyl tetrapeptide-7, dipeptide-2, camellia sinensis leaf extract, punica granatum extract, rubus fruticosus (blackberry) fruit extract, coffea robusta seed extract, vacciunium macrocarpon (cranberry) fruit extract, vitis vinifera (grape) leaf extract, Euterpe oleracea fruit extract, arctostaphylos uva ursi leaf extract, lycium barbarum fruit extract, morinda citrifolia fruit extract, rubus idaeus (raspberry) fruit, brassica oleracea acephala leaf extract, aristotelia chilensis fruit, and/or extract. When any of the above extracts are present within this composition, each extract is present at a concentration ranging from 0.0001 to 2.0 wt % and more preferably from 0.0001 to 1.0 wt %.

As shown in the above table, additional emollients and preservatives may be included within the composition. For example, the emollient may include ethylhexyl isononanoate at a concentration ranging from 0.0001 to 1.5 wt % and more preferably from 0.0001 wt % to 1.0 wt % of the composition. Similarly, preservatives including any combination of ethylhex.ylglycerin, phenoxyethanol, caprylyl glycol and heylene glycol may be included in the composition. When included, each preservative is present at a concentration ranging from 0.1 wt % to 0.75 wt % and more preferably from 0.1 wt % to 0.65 wt %.

In certain aspects, this composition is paraben-free, fragrance-free, and gluten-free.

### Water Based Moisturizer (Facial Applications)

In certain aspects also disclosed is a water-based moisturizer for application to th.e user's face. The water-based moisturizer includes the following: Water, Jojoba Oil, Glycerin, Palmitic Acid, Stearic Acid, Isosorbide Dicaprylate, Butyrospermum Parkii (Shea) Butter, Cetearyl Olivate, Sorbitan Olivate, Cetyl Palmitate, Panthenol, Caprylyl Glycol, 1,2-Hexanediol, Sodium Hyaluronate, Glycyrrhiza Glabra (Licorice) Root Extract, Hydrolyzed Soy Protein, Sodium Citrate, Myristic Acid, Green Tea Extract, Aloe Barbadensis Leaf Extract, and Tropolone.

The moisturizer disclosed herein includes an effective amount of shea butter and jojoba oil. Panthenol (vitamin B5) is included as a moisturizer that further conditions the skin, while hyaluronic acid or a salt thereof is included. The hyaluronic acid or salt thereof is preferably absorbed through the user's skin and as to further plump and/or fill the user's skin by supplementing the user's endogenous collagen and/or extracellular matrix (ECM).

In certain aspects, this composition is paraben-free, fragrance-free, and gluten-free.

### Eye Lid/Eye Area Filler/Plumper

In certain aspects also disclosed is an eye lid/eye area filler/plumping composition that includes the following: Water, Glycerin, Butyrospermum Parkii (Shea) Butter, Himanthalia Elongate Extract, Palmitic Acid, Stearic Acid, Palmitoyl Hexapeptide-12, Cetearyl Olivate, Sorbitan Olivate, Panthenol, Ascorbic Acid (Vitamin C), Bakuchiol, Clycrrhiza Glabra (Licorice) Root Extract, Xanthan Gum, Caprylyl Glycol, 1,2-Hexanediol, Sodium Hyaluronate, Sodium Citrate, Aloe Barbadensis Leaf Extract, Myristic Acid, Fucus Vesiculosus Extract, Saccharomyces Cerevisiae Extract, and Tropolone.

The eye lid/eye area filler/plumping composition includes Panthenol (vitamin B5) is included as a moisturizer that further conditions the skin, while hyaluronic acid or a salt thereof is included. The hyaluronic acid or salt thereof is preferably absorbed through the user's skin and as to further plump and/or fill the user's eye lids by supplementing the user's endogenous collagen and/or extracellular matrix (ECM). In addition, palmitoyl hexapeptide is include, which synergistically acts with the hyaluronic acid or a salt to plump and/or fill the eye lid and/or eye area.

In certain aspects, this composition is paraben-free, fragrance-free, and gluten-free.

### Film Forming Retinol Based Topical Composition

In certain aspects disclosed is a film forming retinol based topical composition having antioxidants. This composition reduces and/or inhibits photodamage from excessive sun exposure and further reduces the appearance of wrinkles, while concurrently reducing pore size. An exemplary embodiment of the film forming retinol based topical composition is provided immediate below.

### Quali-Quantitative Formulation Chart

| | | INGREDIENT | |
|---|---|---|---|
| % (w/w) | | (INCI NAME) | |
| Range | CAS No. | LISTED IN ICID | FUNCTION/s |
| 70.0-90.0 | 541-02-6 | Cyclopentasiloxane | Emollient, Solvent |
| 10.0-20.0 | n/a | Dimethicone | Viscosity controlling |
| | | Crosspolymer | |
| 0.10-0.99 | 25777-71-3 | Methyl | Film forming |
| | | Methacrylate/Glycol | |
| | | Dimetharcylate | |
| | | Crosspolymer | |
| 0.15 | 68-26-8 | Retinol | Skin conditioning |
| 0.10-0.99 | 73398-61-5 | Caprylic/Capric | Skin conditioning |
| | | Triglyceride | |
| 0.10-0.99 | 90106-37-9 | Oryza Sativa | Skin conditioning |
| | | (Rice) Bran Extract | |
| 0.10-0.99 | 84776-03-4 | Helianthus Annuus | Skin conditioning |
| | | (Sunflower) Extract | |
| 0.10-0.99 | 84604-14-8 | Rosmarinus Officinalis | Skin conditioning |
| | | (Rosemary) | |
| | | Leaf Extract | |
| 0.10-0.99 | 1406-18-4 | Tocopherol | Anti-oxidant |

In certain aspects, the film forming retinol based topical composition includes a volatile inorganic solvent that also functions as an emollient that evaporates upon application to a user and further facilitates film formation of the film forming matrix components (discussed further below). In certain aspects, the volatile inorganic solvent is ranges from 65 wt % to 95 wt % of the composition, more preferably from 68 wt % to 92 wt % of the composition, and most preferably from 70 wt % to 90 wt % of the composition. In certain aspects, the volatile inorganic solvent is siloxane based, and more preferably a cyclic siloxane. For example, cyclic siloxanes generally exhibit good solvation and volatility characteristics in siloxanes, and their participation in matrix formation is generally relatively low due to the fact that all silane oxygen atoms are unavailable for polymerization. Thus, the siloxane is not included within the film forming matrix (discussed further below) within this composition. As shown in the table above, the cyclic siloxane is preferably cyclopentasiloxane ranging from 65 wt % to 95 wt % of the composition, more preferably from 68 wt % to 92 wt % of the composition, and most preferably from 70 wt % to 90 wt % of the composition. The volatile component (cyclopentasiloxane) is preferably present in amounts such that the volatile component is more than 50 percent evaporated after 15 minutes at one or more temperatures in the range of from about 30° C. (86° F.) to 40° C. (104° F.).

In certain aspects, the film forming retinol based topical composition further includes a film forming matrix comprised of at least one crosslinking agents and reactive acrylic acids (or the salts thereof) (pre-polymers that react and polymerize upon application on the user). These components react with one another to form a film upon application to a person's skin while the volatile inorganic solvent evaporates away from the skin and remains non-reactive with the film forming matrix. For example, in certain aspects, the at least one crosslinking agents and reactive acrylic acids (or the salts thereof) includes a methacrylic acid (or methacrylate) and an acrylic based (or acrylate based) crosspolymer, with the total amount of both ranging from 0.5 wt % to 7.5 wt % of the overall composition or more preferably 1 wt % to 5 wt % of the overall composition. In certain aspects, the methacrylic acid (or methacrylate) and an acrylic based (or acrylate based) crosspolymer are methyl methacrylate and glycol dimethacrylate crosspolymer respectively, with the total amount of both ranging from 0.5 wt % to 7.5 wt % of the overall composition or more preferably 1 wt % to 5 wt % of the overall composition.

It is preferably to control and maintain operable viscosity of the composition during storage at ambient temperature(s) (i.e., storage conditions ranging from 40° F. to 120° F.). In order to do so, viscosity controlling agents are included in the composition. For example, the inclusion of, for example, a dimethicone crosspolymer provides a composition with a thixotropic consistency. The dimethicone crosspolymer is preferably included within the composition at a concentration ranging from 1.5 wt % to 25 wt % of the composition, from 12.5 wt % to 22.5 wt % of the composition, or most preferably from 10 wt % to 20 wt % of the composition. The dimethicone crosspolymer further aids in crosslinking and film formation of the film forming matrix when the composition is applied to an individual and the volatile inorganic solvent (cyclopentasiloxane) evaporates away.

Further included within the film forming retinol based topical composition is a primary antioxidant/primary active agent. The primary antioxidant is retinoic acid, retinol, or a salt thereof at a concentration range from 0.1 wt % to 1 wt %, and most preferably 0.1 wt % to 0.2 wt % of the composition. These ranges are preferred so that adequate migration of the retinoic acid, retinol, or a salt thereof occurs through the film formed (by the film forming matrix) on the skin occurs and is absorbed by the individual's skin from the film. If concentrations of retinoic acid, retinol, or a salt thereof are decreased below 0.1 wt % inadequate migration of the retinoic acid, retinol, or a salt thereof through the film occurs thus resulting in inadequate skin. absorption, which further results in an inadequate inhibition photodamage from excessive sun exposure, reduction of the appearance of wrinkles, reduction of pore size, or any combination thereof. However, if concentrations of retinoic acid, retinol, or a salt thereof are increased above 0.2 wt %, no marked improvement has been observed. In certain aspects, a secondary antioxidant/secondary active agent is further included in the film forming composition. This secondary antioxidant/secondary active agent synergistically acts with the primary antioxidant/primary active agent (retinoic acid, retinol, or a salt thereof) to inhibit photodamage from excessive sun exposure, reduce of the appearance of wrinkles, and/or reduce of pore size thereby improving the appearance of the user's skin. In certain aspects, the secondary antioxidant/secondary active agent is present at a concentration ranging from 0.05 wt % to 1.5 wt % of the composition and more preferably from 0.10 wt % to 1 wt % of the composition. The secondary antioxidant/secondary active agent preferably includes tocopherol or a salt thereof (e.g., tocopherol acetate), which includes at least one, at least two, at least three, or at least all of the following: alpha-tocopherol, beta-tocopherol, gamma-tocopherol, and delta-tocopherol. In certain aspects, the secondary antioxidant/secondary active agent preferably includes tocopherol or a salt thereof at a concentration ranging from 0.05 wt % to 1.5 wt % of the composition and more preferably from 0.10 wt % to 1 wt % of the composition.

In addition to the above, skin conditioners can be included to further soften the user's skin and to make use of the composition more desirable. For example, in certain aspects, at least one of, at least two of, at least three, or all of the following may include: caprylic/capric triglyceride, Rice Bran Extract (Oryza Sativa Bran Extract), Sunflower Extract (Helianthus Anuus Extract), and/or Rosemary extract (Rosmarinus Officinalis Leaf Extract) with each of these extracts, when present, having concentrations ranging from 0.05 wt % to 1.5 wt % of the composition and more preferably from 0.10 wt % to 1 wt % of the composition.

In certain aspects, the film forming retinol based topical composition is paraben-free, fragrance-free, and gluten-free.

Film Forming Vitamin E (tocopherol) Based Topical Composition

In certain aspects disclosed is a film forming vitamin E (tocopherol) based topical composition. This composition reduces and/or inhibits photodamage from excessive sun exposure and moisturizes skin. This composition can further be advantageously used for wound care to minimize and/or inhibit scar tissue formation. An exemplary embodiment of the film forming vitamin E (tocopherol) based topical composition is provided immediate below.

### Quali-Quantitative Formulation Chart

| | | INGREDIENT | |
|---|---|---|---|
| % (w/w) | | (INCI NAME) | |
| Range | CAS No. | LISTED IN ICID | FUNCTION/s |
| 70.0-90.00 | 541-02-6 | Cyclopentasiloxane | Emollient, Solvent |
| 10.0-20.0 | n/a | Dimethicone Crosspolymer | Viscosity controlling |
| 5.10 | 1406-18-4 | Tocopherol | Anti-oxidant |
| 0.10-0.99 | 25777-71-3 | Methyl | Film forming |
| | | Methacrylate/Glycol | |
| | | Dimetharcylate Crosspolymer | |
| 0.10-0.99 | 90106-37-9 | Oryza Sativa (Rice) Bran Extract | Skin conditioning |
| 0.10-0.99 | 8001-21-6 | Helianthus Annuus | Emollient |
| | | (Sunflower) Seed Oil | |
| 0.10-0.99 | 84776-03-4 | Helianthus Annuus | Skin conditioning |
| | | (Sunflower) Extract | |
| 0.10-0.99 | 84604-14-8 | Rosmarinus Officinalis | Skin conditioning |
| | | (Rosemary) Leaf Extract | |

In certain aspects, the film forming vitamin E (tocopherol) based topical composition includes a volatile inorganic solvent that also functions as an emollient that evaporates upon application to a user and further facilitates film formation of the film forming matrix components (discussed further below). In certain aspects, the volatile inorganic solvent is ranges from 65 wt % to 95 wt % of the composition, more preferably from 68 wt % to 92 wt % of the composition, and most preferably from 70 wt % to 90 wt % of the composition. In certain aspects, the volatile inorganic solvent is siloxane based, and more preferably a cyclic siloxane. For example, cyclic siloxanes generally exhibit good solvation and volatility characteristics in siloxanes, and their participation in matrix formation is generally relatively low due to the fact that all silane oxygen atoms are unavailable for polymerization. Thus, the siloxane is not included within the film forming matrix (discussed further below) within this composition. As shown in the table above, the cyclic siloxane is preferably cyclopentasiloxane ranging from 65 wt % to 95 wt % of the composition, more preferably from 68 wt % to 92 wt % of the composition, and most preferably from 70 wt % to 90 wt % of the composition. The volatile component (cyclopentasiloxane) is preferably present in amounts such that the volatile component is more than 50 percent evaporated after 15 minutes at one or more temperatures in the range of from about 30° C. (86° F.) to 40° C. (104° F.).

In certain aspects, the film forming vitamin F (tocopherol) based topical composition further includes a film forming matrix comprised of at least one crosslinking agents and reactive acrylic acids (or the salts thereof) (pre-polymers that react and polymerize upon application on the user). These components react with one another to form a film upon application to a person's skin while the volatile inorganic solvent evaporates away from the skin and remains non-reactive with the film firming matrix. For example, in certain aspects, the at least one crosslinking agents and reactive acrylic acids (or the salts thereof) includes a methacrylic acid (or methacrylate) and an acrylic based (or acrylate based) crosspolymer, with the total amount of both ranging from 0.5 wt % to 7.5 wt % of the overall composition or more preferably 1 wt % to 5 wt % of the overall composition. In certain aspects, the methacrylic acid (or methacrylate) and an acrylic based (or acrylate based) crosspolymer are methyl methacrylate and glycol dimethacrylate crosspolymer respectively, with the total amount of both ranging from 0.5 wt % to 7.5 wt % of the overall composition or more preferably 1 wt % to 5 wt % of the overall composition.

It is preferably to control and maintain operable viscosity of the composition during storage at ambient te.mperature(s) (i.e., storage conditions ranging from 40° F. to 120° F.). In order to do so, viscosity controlling agents are included in the composition. For example, the inclusion of, for example, a dimethicone crosspolymer provides a composition with a thixotropic consistency. The dimethicone crosspolymer is preferably included within the composition at a concentration ranging from 15 wt % to 25 wt % of the composition, from 12.5 wt % to 22.5 wt % of the composition, or most preferably from 10 wt % to 20 wt % of the composition. The dimethicone crosspolymer further aids in crosslinking and film formation of the film forming matrix when the composition is applied to an individual and the volatile inorganic solvent (eyelopentasiloxane) evaporates away.

Further included within the film forming vitamin E (tocopherol) based topical composition is a primary antioxidant/primary active agent. The primary antioxidant is tocopherol or a salt thereof at a concentration range from 0.1 wt % to 7.5 wt %, more preferably 1.5 wt % to 6.0 wt %, and most preferably from 4.5 wt % to 5.5 wt % of the composition. The tocopherol or a salt thereof (e.g., tocopherol acetate), includes at least one, at least two, at least three, or at least all of the following: alpha-tocopherol, beta-tocopherol, gamma-tocopherol, and delta-tocopherol. These ranges are preferred so that adequate migration of the tocopherol or a salt thereof occurs through the film formed (by the film forming matrix) on the skin occurs and is absorbed by the individual's skin from the film. If concentrations of tocopherol or a salt thereof are decreased below 1.5 wt % inadequate migration of the tocopherol or a salt thereof through the film occurs thus resulting in inadequate skin absorption, which further results in an inadequate inhibition photodamage from excessive sun exposure anchor scar reduction/inhibition during wound healing. However, if concentrations of tocopherol or a salt thereof are increased above 7.5 wt %, no marked improvement has been observed above this concentration(s). In certain aspects, no additional antioxidant(s) are included other than vitamin E (tocopherol) within this film forming composition.

In addition to the above, skin conditioners can be included to further soften the user's skin and to make use of the composition more desirable. For example, in certain aspects, at least one of, at least two of, at least three, or all of the following may include: Rice Bran Extract (Oryza Sativa Bran Extract), Sunflower Extract (Helianthus Anuus Extract), Sunflower seed oil (Helianthus Anuus oil), and/or Rosemary extract (Rosmarinus Officinalis Leaf Extract) with each of these extracts, when present, having concentrations ranging from 0.05 wt % to 1.5 wt % of the composition and more preferably from 0.10 wt % to 1 wt % of the composition.

In certain aspects, the film forming retinol based topical composition is paraben-free, fragrance-free, and gluten-free.

The foregoing description provides embodiments of the invention by way of example only. It is envisioned that other embodiments may perform similar functions and/or achieve similar results. Any and all such equivalent embodiments and examples are within the scope of the present invention and are intended to be covered by the appended claims.

What is claimed is:

1. A film forming retinol based topical composition configured to reduce photodamage front excessive sun exposure, reduce wrinkle appearance, reduce pore sin, or any combination thereof comprising:
(a) a volatile incimanic solvent that concurrently functions as an emollient that evaporates upon application to a user and further facilitates film formation of the film forming matrix components;
(b) a film thrilling matrix comprised of at least one crosslinking agent and acrylic acids or salts thereof that are configured to crosslink and form a matrix post-application to the user,
(c) a viscosity controlling agent configured to control and maintain operable viscosity of the composition during storage at ambient temperature(s) ranging from 4.4° C. (40° F.) to 48.8° C. (120° F.); and
(d) a primary antioxidant comprising retinoic acid, retinol, or salts thereof present at effective concentrations:
(i) to reduce photodamage of the user from excessive sun exposure, reduce wrinkle appearance of the user, reduce pore size of the user, or any combination thereof and
(ii) such that the retinoic acid, retinol, or salts thereof migrate through the film formed by the film forming matrix post-application to the user such that the retinoic acid, retinol, or salts thereof is absorbed by a user from the film,

2. The film forming retinol based topical composition of claim 1, wherein the volatile inorganic solvent ranges from 65 wt % to 95 wt % of the composition and comprises a cyclic siloxane.

3. The film forming retinal based topical composition of claim 1, wherein the cyclic siloxane is a cyclopentasiloxane and is configured liar at least 50 percent evaporation 1.5 minutes post application to the user at a temperatures in the range of from about 30° C. (86° F.) to 40° C. (104° F.).

4. The film forming retinal based topical composition of claim 3, wherein the volatile inorganic solvent and film farming matrix react with one another to form a film upon application to skin of the user while the volatile inorganic solvent evaporates away from the skin and remains non-reactive with the film forming matrix.

5. The film farming retinol based topical composition of claim 4, wherein the at least one crosslinking agents and reactive acrylic acids or the salts thereof comprise a methacrylic acid, methacrylate, an acrylic based or acrylate based crosspolymer, and combinations therein and a total amount of the crosslinking agents and reactive acrylic acids both ranging from 0.5 wt % to 7.5 wt % of the overall composition.

6. The film forming retinol based topical composition of claim 5, wherein the at least one crosslinking agents and reactive acrylic acids or the salts thereof comprise methyl methacrylate and glycol dimethacrylate crosspolymer with a total amount of both ranging from 0.5 wt % to 7.5 wt % of the overall composition.

7. The film forming retinol based topical composition of claim 6, wherein the viscosity controlling agent comprises a dimethicone crosspolymer.

8. The film forming retinol based topical composition of claim 7, wherein the dimnethicone crosspolymer is included within the composition at a concentration ranging from 15 wt % to 25 wt % of the composition.

9. Tje film forming retinol based topical composition of claim 8, wherein the retinoic acid, retinal, or salts thereof is present in the composition at a concentration range from 0.1 wt % to 1 wt %.

10. The film forming retinol based topical composition of claim 9, wherein the retinoic acid, retinol, or salts thereof is present in the composition at a concentration range from 0.1 wt % to 0.2 wt %.

11. The film forming retinol based topical composition of claims 9 or 10, wherein the retinoic acid, retinol, or salts thereof.

12. The film forming retinol based topical composition of claim 11, further comprising a secondary antioxidant/secondary active agent configured to synergistically act with the primary antioxidant/primary active agent to inhibit photodamage of the user from. excessive sun exposure, reduce wrinkle appearance of the user, and/or reduce of pore size of the user.

13. The film forming retinol based topical composition of claim 12, herein the secondary antioxidant/secondary active agent is present at a concentration ranging from 0.05 wt % to 1 wt % of the composition.

14. The film forming retinol based topical composition of claim 13, wherein the secondary antioxidant/secondary active agent comprises a tocopherol or a salt thereof.

15. The film forming retinol based topical composition of claim 14, wherein the tocopherol or salt thereof is present at a concentration ranging from 0.05 wt % to 1.5 wt % of the composition.

16. The film forming retinol based topical composition of claim 15 , wherein the tocopherol comprises at least three of alpha-tocopherol, beta-tocopherol, gamma-tocopherol, and delta-tocopherol.

7. The film forming retinol based topical composition of claim 16, further comprising at least three of caprylic/capric triglyceride. Rice Bran Extract (Oryza Saliva Bran Extract), Sunflower Extract (Helianthus Anuus Extract), and/or Rosemary extract (Rosmarinus Officinalis Leaf Extract) at an overall concentration ranging from 0.05 wt % to 1.5 wt % of the composition.

18. The film firming retinol based topical composition of claim 17, wherein the film forming retinol based topical composition is paraben-free, fragrance-free, and gluten-free.
